(19) United States
(12) Patent Application Publication
(10) Pub. No.: US 2008/0281210 A1
(43) Pub. Date: Nov. 13, 2008

(54) Arterial pressure sensing device

(76) Inventors: Anthony I. Nunez, Beachwood, OH (US); Harry D. Rowland, East Peoria, IL (US)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 12/011,501

(22) Filed: Jan. 25, 2008

Related U.S. Application Data
(60) Provisional application No. 60/897,755, filed on Jan. 26, 2007.

(51) Int. Cl. A61B 5/0215

(52) U.S. Cl. 600/486

(57) ABSTRACT

An implantable medical device including a tubular housing defining a passage between a proximal end and a distal end of the housing. The passage providing fluid communication through the housing. A sensing unit is positioned within the passage and coupled to the housing. The sensing unit is configured to sense at least one of a physical, chemical, and physiological parameter within the passage.

### CROSS REFERENCE TO RELATED APPLICATIONS

This application claims the benefit of U.S. Provisional Application No. 60/897,755, filed Jan. 26, 2007, which is hereby incorporated by reference in its entirety.

### BACKGROUND OF THE INVENTION

This disclosure relates generally to an implantable medical device for drawing fluid, such as blood, from and/or introducing fluids into a patient's vascular system and, more particularly, to a medical device configured to measure a patient's blood pressure in addition to drawing blood from and/or introducing blood and/or intravenous drugs or fluids into the vascular system.

As is common practice in the medical industry when treating an ill patient, drugs and other liquid medical substances are introduced into the patient's intravenous system through an arterial line placed into the patient's radial artery. The arterial line allows the practicing physician, as well as other medical practitioners, to administer drugs to the patient and draw blood from the patient in a relatively easy and painless manner.

However, there are certain drawbacks to such a device including, for example, formation of thrombosis at a tip of an insertion needle of the device. To counter act this problem, it is common practice to periodically reposition the arterial line at a different location on the patient's body as well as introduce a new clot free insertion needle periodically, such as once a week. While these steps may be successful in preventing or limiting the formation of thrombosis and other fluid flow complications, these steps may result in patient discomfort as the patient must be repeatedly subjected to needle insertions into his or her vascular system on a frequent basis.

In addition, these devices only allow for the introduction of fluid into the body or extraction of fluid from the body. Further, because the device components are replaced repeatedly, diagnostic data or information cannot be collected.

### BRIEF DESCRIPTION OF THE INVENTION

In one aspect, an implantable medical device is provided. The implantable medical device includes a tubular housing defining a passage between a proximal end and a distal end of the housing. The passage provides fluid communication through the housing. A sensing unit is positioned within the passage and coupled to the housing. The sensing unit is configured to sense at least one of a physical, chemical, and physiological parameter within the passage.

In another aspect, an arterial pressure sensor device is provided. The arterial pressure sensor device includes a tubular housing defining a passage between a proximal end and a distal end of the housing. The passage provides fluid communication through the housing. A sensing unit is positioned within the passage and coupled to the housing. The sensing unit is configured to sense a pressure within the passage corresponding to an arterial pressure.

In another aspect, an implantable medical device is provided. The implantable medical device includes a tubular conduit defining a passage between a proximal end and a distal end of the conduit. The passage provides fluid communication through the conduit. A sensing unit is positioned within the passage and coupled to the conduit. The sensing unit includes at least one sensor configured to sense at least one of a physical, chemical, and physiological parameter to facilitate obtaining diagnostic data. The sensing unit is configured to transmit the diagnostic data wirelessly to an external receiver.

In another aspect, a method for fabricating an implantable medical device is provided. The method includes fabricating a tubular housing defining a passage between a proximal end and a distal end of the housing; positioning a sensing unit within the passage; and coupling the sensing unit to the housing. The sensing unit is configured to sense at least one of a physical, chemical, and physiological parameter within the passage.

In another aspect, an implantable device is provided. The implantable device includes a housing including a first sheet of material having a first surface defining a void therein. An electrically conducting surface is patterned in the void formed in the first surface. A second sheet of material is sealingly coupled to the first surface to enclose the void and form a hermetically sealed cavity. The second sheet of material is patterned with electrically functional components and sealingly coupled to the first surface to form the housing defining a passage and a sensor configured to sense at least one of a physical, chemical, and physiological parameter within the passage.

### BRIEF DESCRIPTION OF THE DRAWINGS

FIG. 1 is a side view of a housing for an exemplary arterial pressure sensing device;

FIG. 2 is a top view of the housing shown in FIG. 1;

FIG. 3 is a perspective view of the housing shown in FIG. 1;

FIG. 4 is a side view of an exemplary arterial pressure sensing device including a sensor unit positioned within the housing shown in FIGS. 1-3;

FIG. 5 is a top view of the arterial pressure sensing device shown in FIG. 4;

FIG. 6 is a perspective view of the arterial pressure sensing device shown in FIG. 4;

FIG. 7 is an exploded perspective view of the arterial pressure sensing device shown in FIG. 4;

FIG. 8A is a top view of a first sheet of material for forming a housing as shown in FIG. 11 including a sensor positioned at each end of the housing;

FIG. 8B is a cross-sectional view of the first sheet of material shown in FIG. 8A taken along A-A in FIG. 8A;

FIG. 9A is a top view of the first sheet of material shown in FIG. 8A and including a electrically conducting surface;

FIG. 9B is a cross-sectional view of the first sheet of material shown in FIG. 9A taken along B-B in FIG. 9A;

FIG. 10A is a top view of a second sheet of material for forming a housing as shown in FIG. 11;

FIG. 10B is a cross-sectional view of the second sheet of material shown in FIG. 10A taken along C-C in FIG. 10A;

FIG. 11 is a perspective view of a housing including a sensor positioned at each end of the housing;

FIG. 12 is a side sectional view of a sensor configured for positioning within a housing;

FIG. 13 is a side sectional view of the housing shown in FIG. 12 with a sensor deployed at an end of the housing;

FIG. 14 is an exploded perspective side view of an alternative exemplary implantable medical device including a sensing unit positioned within a housing; and

FIG. 15 is an exploded perspective top view of the implantable medical device shown in FIG. 14.

### DETAILED DESCRIPTION OF THE INVENTION

The embodiments described herein provide an implantable medical device and a method to facilitate measuring arterial blood pressure of a patient in a critical, acute, or sub-acute environment. The device also allows the practitioner to limit pain to which the patient is subjected, as well as obtain diagnostic data or information to assist in directing patient care including making decisions regarding administration of medication, such as blood pressure medication, for example.

In one embodiment, the device includes a disposable sensing unit and a housing, which allows external connection of tubing for fluid introduction and/or extraction. The sensing unit includes one or more sensors that are positioned within a housing passage and configured to sense or measure physical, chemical and/or physiological parameters or variables to facilitate obtaining data for arterial pressure analysis, temperature analysis, blood chemical analysis, blood osmolar analysis, and cellular count analysis, for example. The sensing unit is configured to transmit the measurement data wirelessly to an external receiver. Each sensor may be a pressure sensor, an optical sensor, a biochemical sensor, a protein sensor, a motion sensor (e.g., an accelerometer or a gyroscope), a temperature sensor, a chemical sensor (e.g., a pH sensor), a biochemical sensor, or a genetic sensor, for example. In one embodiment, the device includes a plurality of pressure sensors that are fabricated using a suitable microelectromechanical systems (MEMS) technology that utilizes a resonating frequency of an LC resonator. In alternative embodiments, the device includes a plurality of sensors that function as capacitive, piezoelectric or piezoresistive sensors.

The housing defines a passage having a suitable cross-sectional area to facilitate drawing or extracting fluid, such as blood, from a patient's vascular system and/or introducing fluid, such as blood, drugs and/or medical substances, into the patient's vascular system. The housing may include a plug configured to prevent or limit fluid leakage from the patient's vascular system. In one embodiment, the plug may include one or more sensors. The plug may be positioned at either opening of the passage or at a suitable position within the passage to limit fluid leakage and/or sense at least one parameter. Further, the housing may include a suitable connection assembly that allows external tubes to be connected to the housing for drawing fluid from and/or introducing fluid into the patient's vascular system.

In one embodiment, the housing is positioned within and/or anchored into the patient's tissue and against or through a vessel wall using a suitable process including, without limitation, an adhesion anchoring process and/or a mechanical anchoring process. In a particular embodiment, the housing includes a distal retainer and an opposing proximal retainer that is positioned with respect to an internal surface of a vessel wall and an external surface of the patient's skin, respectively. The distal retainer and the proximal retainer subsequently are coupled together to anchor the housing at least partially within the patient's tissue. Alternatively, the housing is coupled in position using a medically safe adhesive and/or tissue in-growth onto an outer surface of the housing.

Although the following disclosure describes a sensor that is configured to measure and/or monitor an arterial blood pressure within the vessel to facilitate obtaining data for blood pressure analysis, it should be apparent to those skilled in the art and guided by the teachings herein provided that the sensor as described herein may be configured to measure one or more physical, chemical, and/or physiological parameters or variables to facilitate obtaining data for temperature analysis, blood chemical analysis, blood osmolar analysis, and cellular count analysis, for example.

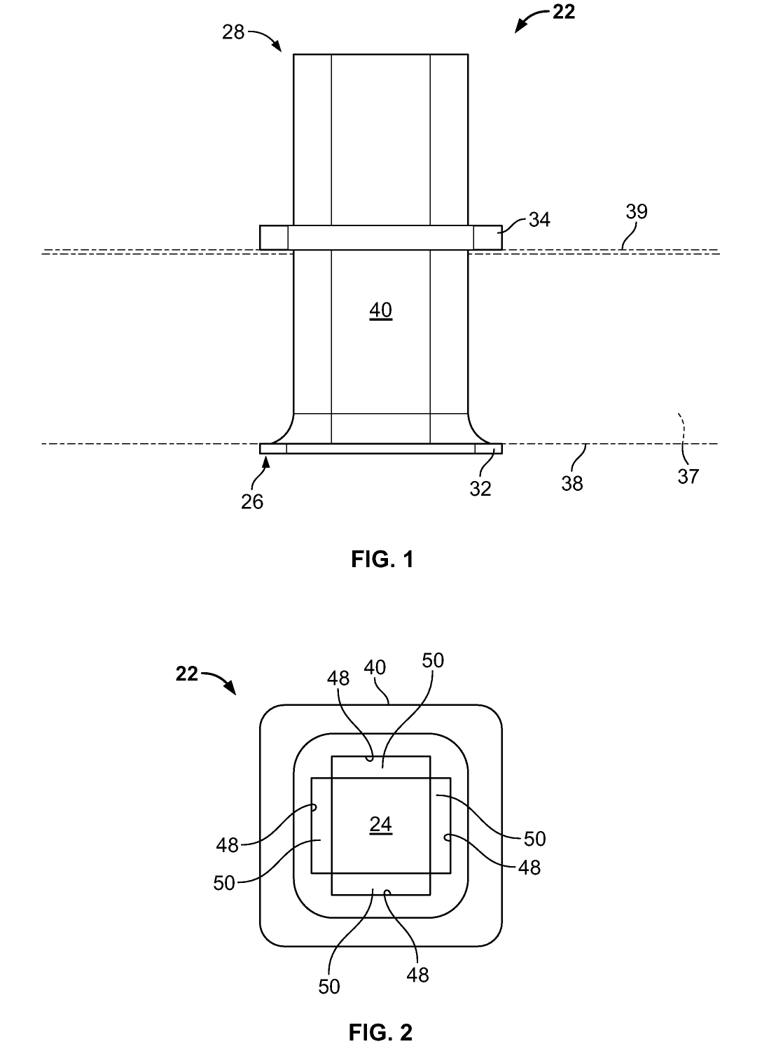

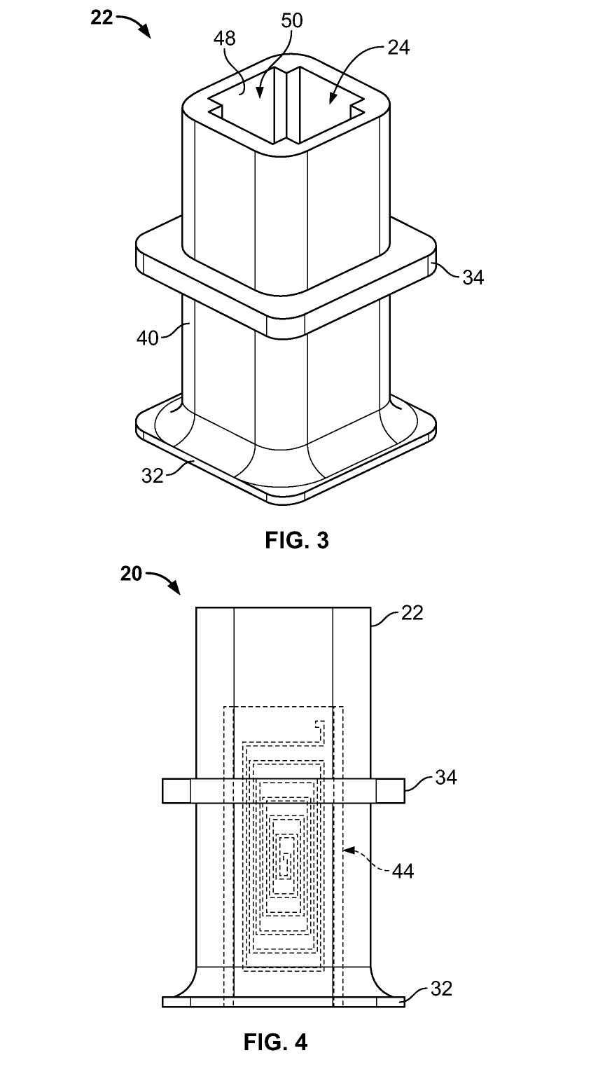

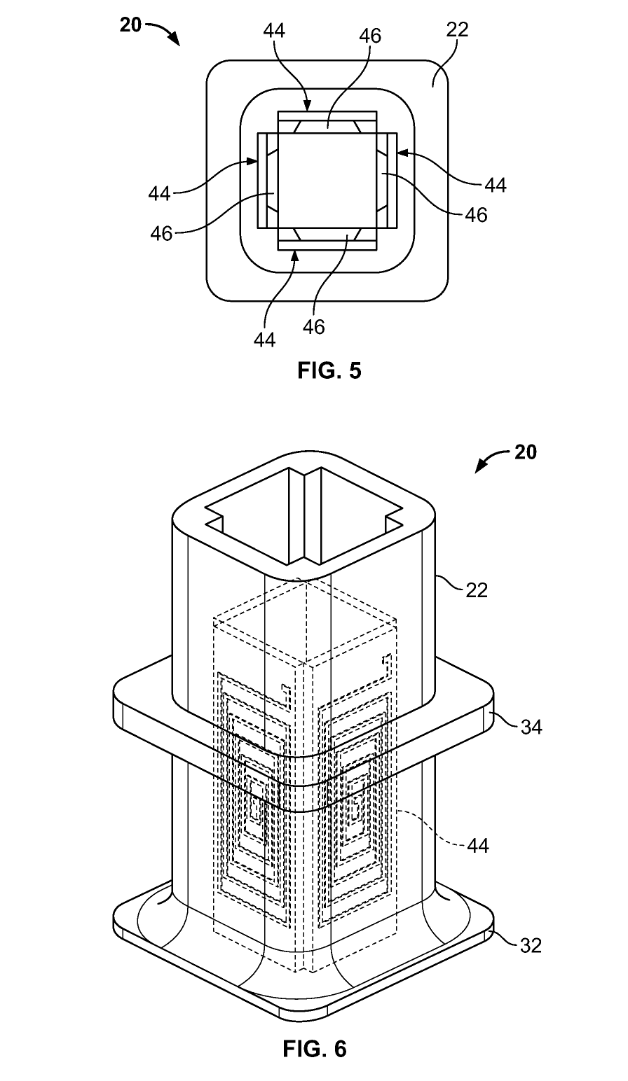

As shown in FIGS. 1-7, an implantable medical device, such as an arterial pressure sensing device 20, includes a tubular housing 22. Referring further to FIGS. 1-4, housing 22 is fabricated from any suitable biocompatible material including, without limitation, a suitable biocompatible metal, alloy, composite, plastic or polymeric material. Housing 22 defines a passage 24 that extends between a proximal end 26 and an opposing distal end 28 of housing 22. In one embodiment, as shown in FIG. 2, for example, passage 24 has a general square cross-sectional area. Alternatively, passage 24 may have any suitable cross-sectional area including, without limitation, a circular, hexagonal, octagonal or other polygonal or non-polygonal cross-sectional area. Further, passage 24 has suitable dimensions to provide fluid communication through housing 22 to facilitate drawing or extracting fluid, such as blood, from a patient's vascular system and/or introducing fluid, such as blood, drugs and/or medical substances, into the patient's vascular system, as well as connecting suitable external tubing to housing 22 for fluid extraction and/or introduction, as desired.

In one embodiment, housing 22 includes a first or proximal retainer 32 formed on or initially coupled to housing 22 at or near proximal end 26 and/or a second or distal retainer 34 formed on or coupled to housing 22 at or near a midsection 36 of housing 22 or distal end 28 of housing 22. Housing 22 is positioned at least partially within the patient's tissue 37, shown schematically in phantom lines in FIG. 1, such that proximal end 26 contacts a skin surface 38 and distal retainer 34 contacts a wall of the vessel 39, shown schematically in a phantom line in FIG. 1, to retain housing 22 properly positioned within tissue 37. In a particular embodiment, distal retainer 34 is positioned within a vessel, such as an artery, and contacts an inner surface of a wall of the vessel, as shown in FIG. 1. With distal retainer 34 positioned within the vessel, housing 22 provides fluid communication between the vessel and a medical device or instrument, such as a needle or catheter positioned within passage 24 or a tubing operatively coupled to proximal end 26 of housing 22.

In one embodiment, proximal retainer 32 is movable with respect to distal retainer 34, such as slidably movable along a length of housing 22 with respect to distal retainer 34 and/or rotationally movable about a perimeter and along the length of housing 22, to facilitate retaining housing 22 properly positioned within the tissue. In a particular embodiment, a suitable snapping mechanism, such as a plurality of teeth (not shown) formed on an outer surface 40 of housing 22 and at least one cooperating tooth (not shown) formed on proximal retainer 32, allows proximal retainer 32 to be adjustably positioned with respect to distal retainer 34 and secured in proper position. Proximal retainer 32 is adjustably positioned with respect to distal retainer 34 to apply a suitable compressive force on tissue 37 to retain housing 22 properly positioned within tissue 37. Further, such compressive force may promote sealing of the insertion site about housing 22 to prevent or limit bleeding and/or fluid leakage. Alternatively or in addition, a screw mechanism, such as a helical thread (not shown) formed on outer surface 40 and a cooperating helical thread (not shown) formed on proximal retainer 32, allows proximal retainer 32 to be adjustably positioned with respect to distal retainer 34.

In an alternative embodiment, housing 22 is affixed to tissue 37 to retain housing 22 properly positioned within tissue 37 using a suitable biocompatible adhesive including, without limitation, an acrylic-based adhesive, such as cyanoacrylate, an epoxy-based adhesive, a polyurethane-based adhesive, and/or a silicon-based adhesive, such as organopolysiloxane.

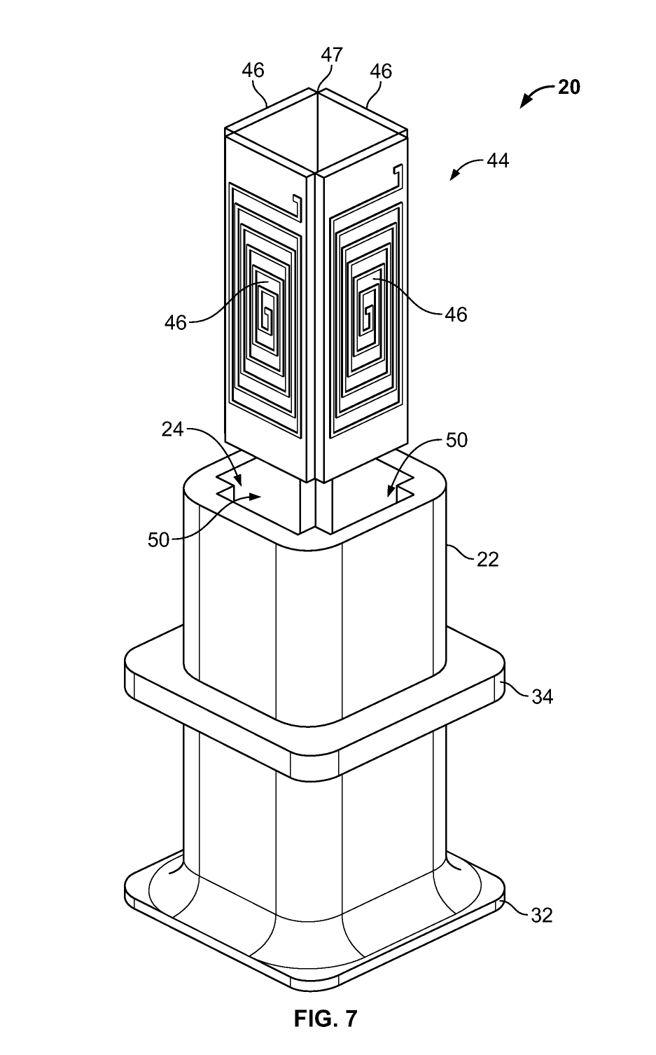

In an exemplary embodiment, one or more sensors are operatively coupled, directly or indirectly, to or incorporated within device 20. Referring further to FIGS. 4-7, housing 22 is configured to receive a sensing unit 44 including one or more sensors 46 configured to sense or measure one or more physical, chemical, and/or physiological parameters or variables to facilitate obtaining data for arterial pressure analysis, temperature analysis, blood chemical analysis, blood osmolar analysis, and cellular count analysis, for example, and transmit the measurement data wirelessly to an external receiver. Sensor 46 may be a pressure sensor, an optical sensor, a biochemical sensor, a protein sensor, a motion sensor (e.g., an accelerometer or a gyroscope), a temperature sensor, a chemical sensor (e.g., a pH sensor), or a genetic sensor, for example. In one embodiment, sensing unit 44 includes a substrate 47, as shown in FIG. 7, such as a polysilicon substrate or other suitable material, to which each sensor 46 is coupled. In alternative embodiments, one or more sensors 46 are positioned at one or more suitable locations or surfaces on housing 22 and/or a plug, as described below. Further, the plug can be coupled to housing 22 at any suitable location on device 20, such as on housing 22.

In one embodiment, each sensor 46 is coupled to an inner surface 48 of housing 22, as shown in FIGS. 4-6, and positioned at a device/fluid flow interface. Sensors 46 are arranged on inner surface 48 such that passage 24 provides desirable fluid communication through housing 22. In an alternative embodiment, sensing unit 44 and/or each sensor 46 is integrated with housing 22 such that sensor 46 is capable of providing high fidelity readings or measurements of intravascular pressure, for example. Referring to FIG. 2, in one embodiment, one or more voids or wells 50 are defined within inner surface 48 of housing 22 to accommodate a corresponding sensor 46 of sensing unit 44. More specifically, void 50 is formed in inner surface 48 having a shape and configuration corresponding to a shape and configuration of corresponding sensor 46 to facilitate coupling or integrating sensor 46 with housing 22. In one embodiment, sensors 46 are fabricated separately from housing 22, and subsequently affixed to housing 22. Alternatively, sensors 46 and housing 22 are fabricated as an integrated device using a suitable technology, such as a MEMS technology. In a further alternative embodiment, housing 22 is configured as a sensor having sensing capabilities.

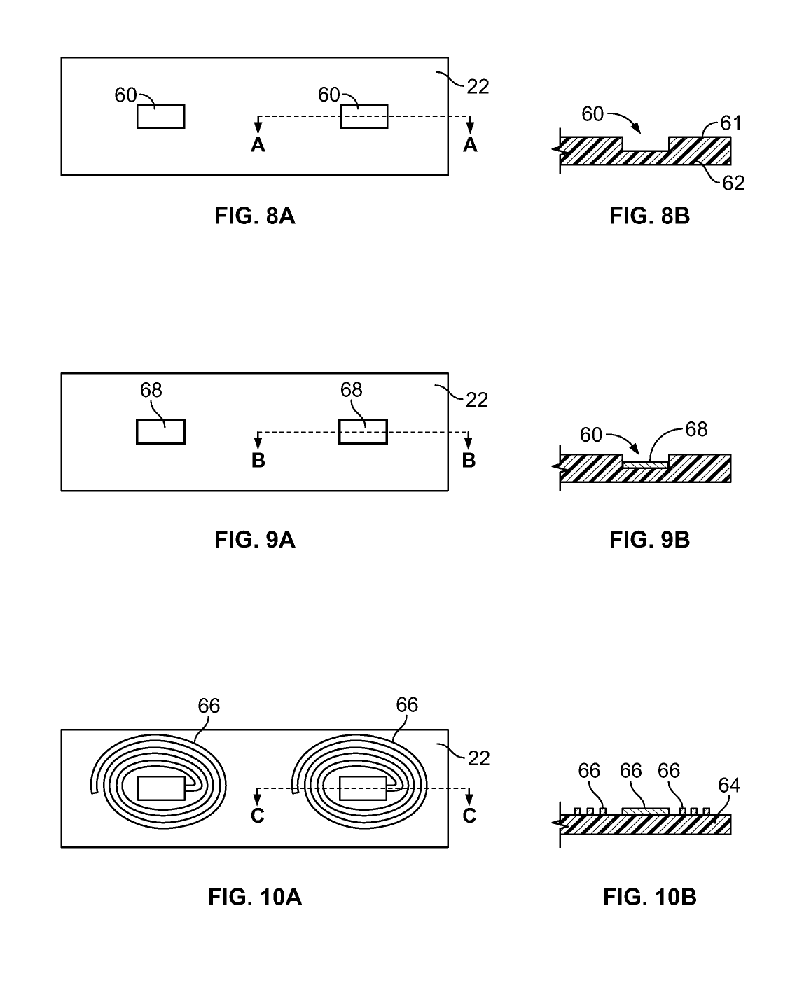

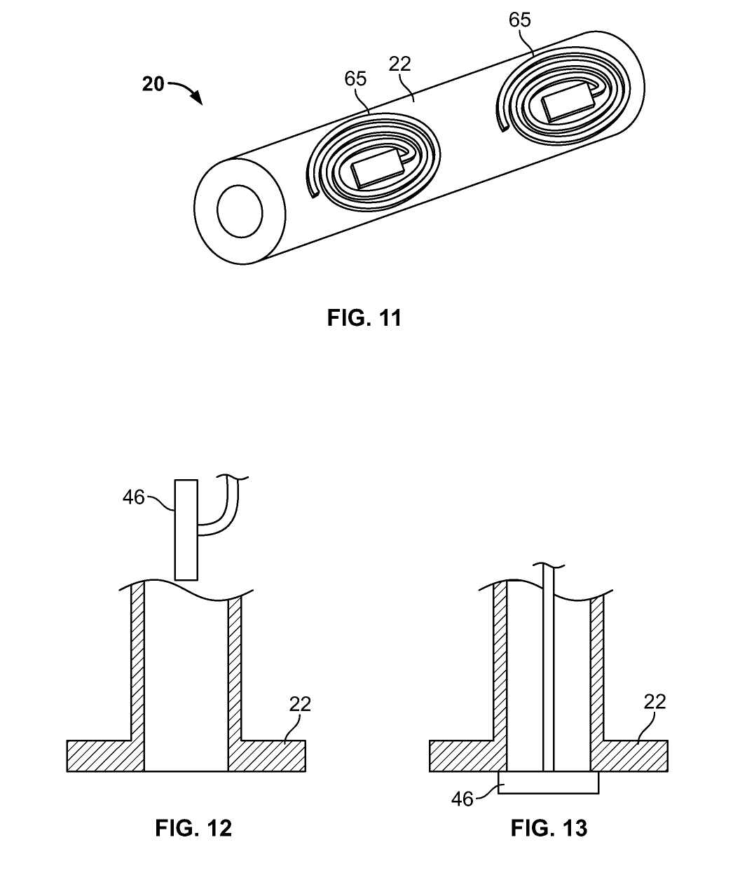

Referring to FIGS. 8A-11, in one embodiment, housing 22 may be configured with sensing capabilities. In a particular embodiment, housing 22 includes a wireless capacitive pressure sensor having pressure sensing capabilities. A void 60, as shown in FIG. 8B, is formed on a first surface 61 of a first sheet or layer of material 62 including, without limitation, a suitable polymer, silicon, or fused silica material. A second sheet or layer of material 64, as shown in FIG. 10B, is coupled to first surface 61 to enclose void 60, thus forming a hermetically sealed cavity 65 in housing 22. Second sheet 64 is fabricated of any suitable material including, without limitation, a polymer, silicon, or fused silica material. In a particular embodiment, second sheet 64 is patterned with electrically functional components 66, such as conductors, dielectrics, capacitors, resistors, and/or semiconductors. The components are patterned or deposited using suitable semiconductor fabrication techniques and/or other suitable printed electronics techniques known to those skilled in the art and guided by the teachings herein provided. In one embodiment, the components are formed by depositing a conducting seed layer, depositing a photoresist on the seed layer, patterning the photoresist, and electroplating components on the exposed regions of the seed layer. Additional portions of housing 22 may also be patterned with electrically functional components. For example, an electrically conducting surface 68 is patterned in void 60 formed in first surface 61 and partially forming cavity 65. In one embodiment, electrically conducting surface 68 forms one surface of a capacitor plate. As shown in FIG. 11, first sheet 62 is sealingly coupled to second sheet 64 to form housing 22 defining passage 24 with one or more sensors 46 positioned at opposing end portions of housing 22 to sense and/or monitor for stenosis, for example.

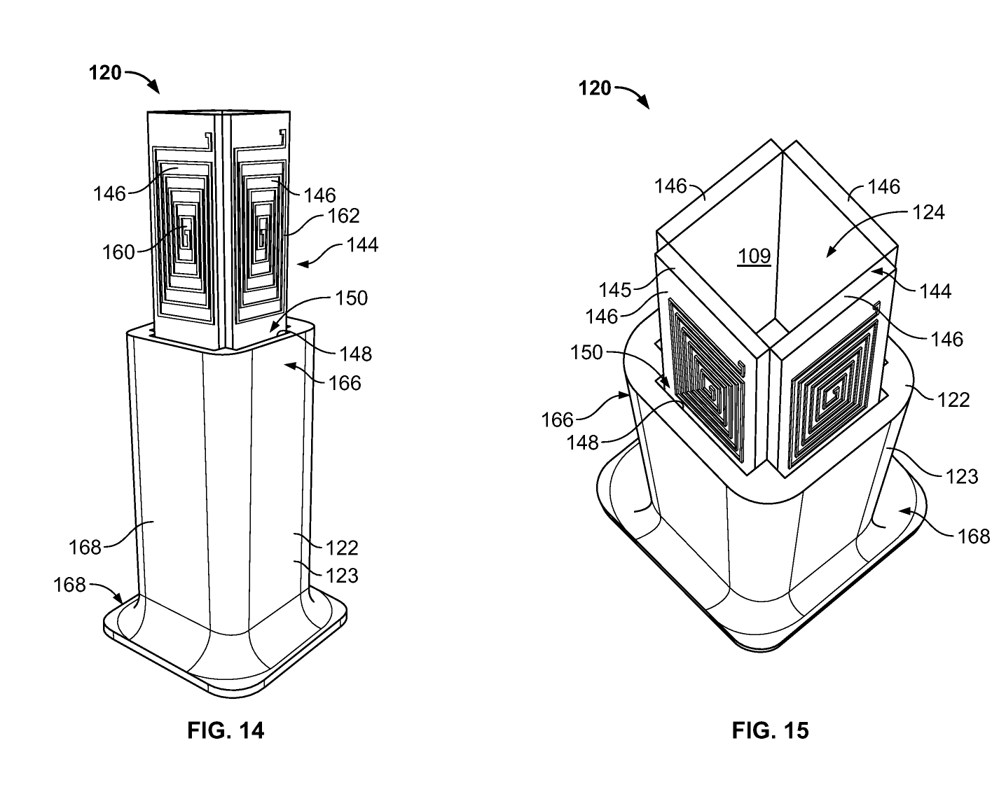

Referring to FIGS. 12 and 13, in an alternative embodiment, a wired or wireless sensor 46 is configured to be positioned within housing 22 and deployed at an end of housing 22 to temporarily prevent fluid flow through passage 24 to monitor a pressure. In further embodiments, sensor 46 may have an opening or a valve formed through sensor 46 to allow extraction and/or insertion of a needle or fluids, for example. Additionally, sensor 46 may be coupled to the opposing end of housing 22, such as attached or screwed to the opposing end of housing 22, to temporarily prevent or stop fluid flow through passage 24 to monitor the pressure.

It should be apparent to those skilled in the art and guided by the teachings herein provided that sensor 46 may have any suitable dimension such that device 20 functions as described herein. In a particular embodiment, sensing unit 44 and/or sensor 46 has a suitable size and/or configuration such that laminar blood flow, rather than turbulent blood flow, is sensed and/or monitored. Further, sensing unit 44 and/or each sensor 46 may have a suitable shape and/or profile corresponding to a shape and/or profile of inner surface 48 to facilitate coupling or integrated sensing unit 44 with housing 22.

Sensing unit 44 is coupled to or integrated with housing 22 during or after fabrication of housing 22. In one embodiment, sensing unit 44 is coupled to housing 22 using a suitable process to minimize restriction, obstruction, and/or occlusion of fluid flow through passage 24 with sensing unit 44 positioned within passage 24. Referring to FIGS. 4-6, each sensor 46 of sensing unit 44 is coupled to housing 22 using a suitable biocompatible material. In one embodiment, sensor 46 is coupled within corresponding void 50 to inner surface 48 using a suitable biocompatible adhesive including, without limitation, an acrylic-based adhesive, such as cyanoacrylate, an epoxy-based adhesive, a polyurethane-based adhesive, and/or a silicon-based adhesive, such as organopolysiloxane. In an alternative embodiment, sensing unit 44 is retained in proper position within housing 22 using a suitable snapping mechanism or threaded mechanism. In further alternative embodiments, sensor 46 is coupled to housing 22 using a suitable mechanism and/or process known to those skilled in the art and guided by the teachings herein provided including, without limitation, a chemical bonding process, a heat bonding process, a soldering process, a suturing process using a non-absorbable suture, or an outer packaging material. In a particular embodiment, the outer packaging material is chemically treated with a suitable heparin-bonded ePTFE material, a drug eluting material that inhibits cellular overgrowth, such as Tacrolimus or Sirolimus, or another suitable anti-metabolite to provide an anti-thrombotic outer surface.

In one embodiment, device 20 includes a plurality of pressure sensors 46 that are fabricated using a suitable microelectromechanical systems (MEMS) technology. In a particular embodiment, sensor 46 is fabricated using a MEMS technology that utilizes a resonating frequency of an LC Tank circuit or a suitable capacitive or piezoelectric technology to measure pressure within passage 24. Sensor 46 is configured to facilitate transmission of data wirelessly to an external device, such as a user-controlled receiver. In a biomedical application, the signal is desirably transmitted through the patient's surrounding tissue without distorting or lowering a strength of the signal such that the signal is lost or undecipherable.

In a particular embodiment, sensor 46 includes a capacitance inductor circuit arranged in a parallel configuration to form an LC tank circuit. The LC tank circuit generates resonating frequency signals that are emitted through housing 22 and transmitted to an at least partially external device, such as a patient signaling device, wherein the signals are processed and deciphered. Based on the transmitted signals, the external device generates an output representative of an internal pressure of the vessel, for example. More specifically, in one embodiment, sensor 46 is configured to sense fluid flow through passage 24 and generate a signal representative of a fluid pressure within passage 24 to facilitate measuring and/or monitoring blood pressure, for example. Sensor 46 generates a signal representative of a fluid pressure within the vessel. It should be apparent to those skilled in the art and guided by the teachings herein provided that sensor 46 may be fabricated using any suitable technology and/or process. In alternative embodiments, device 20 includes a plurality of sensors 46 including a capacitive pressure sensing device, a piezoelectric pressure sensing device or a piezoresistive pressure sensing device.

In one embodiment, sensing unit 44 includes a suitable pressure sensor 46, which operates through a displacement of two capacitor plates that are connected in parallel to an inductor. As fluid moves through passage 24 a pressure is induced on one or both capacitor plates. This pressure displaces the capacitor plate(s) and subsequently changes the capacitance value of sensing unit 44. The resonating frequency emitted from sensing unit 44 is a function of the inductance and capacitance values seen in the circuitry. Because the capacitance values of the circuitry changes with the changing internal pressure of the patient's artery, the subsequently emitted resonating frequency will change with the changing pressure. This shift in the resonating frequency can be read through an external receiver unit and deciphered to generate an internal pressure reading within the patient's artery.

In one embodiment, sensor 46 is coated with at least one biocompatible material including, without limitation, one or more suitable biocompatible polymers such as a slow release polymer impregnated with an anti-metabolite inhibiting in-tissue growth. In a particular embodiment, at least a portion of sensor 46 is coated with a drug eluting material that prohibits in-tissue growth on sensor 46.

In one embodiment, sensing unit 44 is removable from within housing 22, for example, if thrombosis or other material buildup within passage 24 is detected, such as at an opening to passage 24 at proximal end 26 and/or at an opening to passage 24 at distal end 28. Upon removing sensing unit 44 from within housing 22, sensing unit 44 can be cleaned and reinserted or used sensing unit 44 can be disposed of and replaced with a new sensing unit 44. Further, device 20 includes a suitable plug (not shown) configured to couple to housing 22 at proximal end 26 to prevent or limit leakage from device 20. Additionally, device 20 may include a connection assembly configured to couple external tubes to housing 22 for drawing blood from and/or introducing fluid into the patient's vascular system. In one embodiment, sensing unit 44 is coupled to housing 22 using a suitable snapping or screwing mechanism for long-term installment of sensing unit 44 within housing 22. In a further embodiment, housing 22, sensing unit 44 and/or sensors 46 are coated in a biocompatible material for long-term use.

Referring to FIGS. 14 and 15, in one embodiment, an implantable medical device 120 includes a tubular conduit 122, such as a bypass graft, defining a passage 124 between a proximal end and a distal end of conduit 122. In one embodiment, conduit 122 includes an outer graft wall 123 made of a suitable polymer or dacron material. In one embodiment, as shown in FIGS. 14 and 15, for example, passage 124 has a general square cross-sectional area. Alternatively, passage 124 may have any suitable cross-sectional area including, without limitation, a circular, hexagonal, octagonal or other polygonal or non-polygonal cross-sectional area. Further, passage 124 has suitable dimensions to provide fluid communication through conduit 122.

In an exemplary embodiment, one or more sensors are operatively coupled, directly or indirectly, to or incorporated within device 120. Referring further to FIGS. 14 and 15, conduit 122 is configured to receive a sensing unit 144 including one or more sensors 146 configured to sense or measure one or more physical, chemical, and/or physiological parameters or variables to facilitate obtaining data for arterial pressure analysis, temperature analysis, blood chemical analysis, blood osmolar analysis, and cellular count analysis, for example, and transmit the measurement data wirelessly to an external receiver. Sensor 146 may be a pressure sensor, an optical sensor, a biochemical sensor, a protein sensor, a motion sensor (e.g., an accelerometer or a gyroscope), a temperature sensor, a chemical sensor (e.g., a pH sensor), or a genetic sensor, for example. In one embodiment, sensing unit 144 includes a substrate, such as a polysilicon substrate, to which each sensor 146 is coupled.

As shown in FIGS. 14 and 15, sensing unit 144 is inserted into passage 124 using a suitable process, such as a suitable endovascular process. Sensing unit 144 may have any suitable shape and/or configuration corresponding to a shape and/or configuration of an inner surface of conduit 122. Sensing unit 144 is positioned within passage 124 and coupled to conduit 122 such that passage 124 provides desirable fluid flow through conduit 122.

In one embodiment, each sensor 146 is coupled to an inner surface 148 of conduit 122, as shown in FIGS. 14 and 15, and positioned at a device/fluid flow interface. Sensors 146 are arranged on inner surface 148 such that passage 124 provides desirable fluid communication through conduit 122. In an alternative embodiment, sensing unit 144 and/or each sensor 146 is integrated with conduit 122 such that sensor 146 is capable of providing high fidelity readings or measurements of intravascular pressure, for example. Referring to FIGS. 14 and 15, in one embodiment, one or more voids or wells 150 are defined within inner surface 148 of conduit 122 to accommodate a corresponding sensor 146 of sensing unit 144. More specifically, void 150 is formed in inner surface 148 having a shape and configuration corresponding to a shape and configuration of corresponding sensor 146 to facilitate coupling or integrating sensor 146 with conduit 122. In one embodiment, sensors 146 are fabricated separately from conduit 122, and subsequently affixed to conduit 122. Alternatively, sensors 146 and conduit 122 are fabricated as an integrated device using a suitable technology, such as a MEMS technology. In a further alternative embodiment, conduit 122 is configured as a sensor having sensing capabilities.

Each sensor 146 of sensing unit 144 is coupled to conduit 122 using a suitable biocompatible material. In one embodiment, sensor 146 is coupled within corresponding void 150 to inner surface 148 using a suitable biocompatible adhesive including, without limitation, an acrylic-based adhesive, such as cyanoacrylate, an epoxy-based adhesive, a polyurethane-based adhesive, and/or a silicon-based adhesive, such as organopolysiloxane. In an alternative embodiment, sensing unit 144 is coupled to inner surface 148 using a suitable snapping mechanism or threaded mechanism. In further alternative embodiments, sensor 146 is coupled to conduit 122 using a suitable mechanism and/or process known to those skilled in the art and guided by the teachings herein provided including, without limitation, a chemical bonding process, a heat bonding process, a soldering process, a suturing process using a non-absorbable suture, or an outer packaging material. In a particular embodiment, the outer packaging material is chemically treated with a suitable heparin-bonded ePTFE material, a drug eluting material that inhibits cellular overgrowth, such as Tacrolimus or Sirolimus, or another suitable anti-metabolite to provide an anti-thrombotic outer surface.

It should be apparent to those skilled in the art and guided by the teachings herein provided that sensor 146 may have any suitable dimension such that device 120 functions as described herein. In a particular embodiment, sensing unit 144 and/or sensor 146 has a suitable size and/or configuration such that laminar blood flow, rather than turbulent blood flow, is sensed and/or monitored. Further, sensing unit 144 and/or each sensor 146 may have a suitable shape and/or profile corresponding to a shape and/or profile of inner surface 148 to facilitate coupling or integrated sensing unit 144 with conduit 122.

As shown in FIG. 14, in one embodiment, each sensor 146 includes a capacitor plate 160 and a coil 162. Sensor 146 is fabricated from a suitable substrate, such as a polymer material or a silicon material utilizing techniques known to those skilled in the art and guided by the teachings herein provided, such as a suitable MEMS technology.

In a particular embodiment, sensors 146 are positioned on inner surface 148 and/or an opposing outer surface of conduit 122 at equal or varying intervals along a length of conduit 122 to facilitate obtaining measurement data at various intervals for comparative measurements and analysis. For example, conduit 122, configured for use as a vascular access graft, typically forms one or more areas of excessive cellular growth at a proximal arterial portion 166 and/or a distal venous portion 168 of device 120. Sensors 146 placed at proximal arterial portion 166 and/or a distal venous portion 168 allows monitoring of cellular overgrowth and stenosis as the pressure and flow differential characteristics change. This monitoring can detect a positive change or a negative change. Further, a rate of change can also be an indicator as well.

Sensing unit 144 may be positioned within conduit 122 as a independent or separate unit during a procedure for implanting conduit 122 in the patient to allow measurement and trimming of conduit 122 prior to surgical implantation. Typically, before or during the procedure, adjustments to the dimensions of conduit 122, such as a length of conduit 122, are necessary due to a natural variation in a distance between attachment points and considerations for curvatures in each recipient. In one embodiment wherein conduit 122 is fabricated of a semirigid material, conduit 122 provides a near ideal surface to glue, attach by force, sew or stent sensor 146 to various points along the conduit length.

In a particular embodiment as shown in FIG. 15, edges 145 of sensing unit 144 are tapered to create a low profile edge to prevent or reduce turbulence as fluid flows through conduit 122. Further, sensor 146 is positioned at or near inner surface 148 of conduit 122 such that sensor 146 senses or measures the laminar aspect of the fluid flow rather than the turbulent aspect of the fluid flow.

In one embodiment, a method for treating cardiovascular disease including the measurement and monitoring of blood pressure is provided. A pressure signal is generated that is indicative of the fluid pressure within the arterial and/or venous system of the human body. The delivery of pharmaceutical therapy is controlled based at least partially on the pressure signal. The pressure signal is transmitted to a patient signaling device located at least partially externally to the patient. The patient signaling device processes the signal and transmits instructive treatment signals that are based at least partially on the processor output that will guide the patient and physician in determining a change in therapy.

In a further embodiment, a method is provided for withdrawing blood from the housing unit in a manner that facilitates continuous monitoring of the pressure parameters. This allows for the attachment of a syringe to the housing unit with a one way valve controlling the flow of blood out of the housing unit. The housing unit is coated with an anticoagulant within the inner passage to prevent clot formation. In one embodiment, the coating is a polymer-based material, such as ePTFE (expanded polytetrafluoroethylene) that is covalently bonded to unfractionated heparin or its derivatives. The polymer-based material may also be a slow release polymer that is impregnated with Tacrolimus and/or Sirolimus and/or an antimetabolite that inhibits tissue in-growth.

In one embodiment, a method is provided for calibrating the measured pressure against external atmospheric pressure such that the adjusted pressure signal is based in part upon the signal sensor and the obtained atmospheric pressure.

In one embodiment, calibration of the device is initiated at initial manufacture and then at the time of implantation. The device coil is calibrated to a unique frequency signature of the device just prior to implantation with the reader set at atmospheric pressure based on a sea level height at which the procedure is taking place. Once zeroed and deployed, the sensor can then be recalibrated periodically by comparative measurement with standard blood pressure cuff readings. The recalibration of the device can also be performed by ultrasound interrogation. The piezoelectric signal and frequency shift generated by the deflection membrane as a prescribed set ultrasound frequency change can be used to determine a degree of membrane damping occurring as a result of cellular and non-cellular deposition.

In one embodiment, wherein the device includes a plurality of sensors, calibration includes placement of a reference sensor as one of the sensors. The reference sensor provides the ability to internally have affixed a reference point within the blood stream. The capacitance of the reference sensor will change only as a degree of cellular and non-cellular material deposit over time.

The reference sensor allows for calibration in addition to external calibration and accounts for drift in the signal over time based on a change in the materials as they are infiltrated and changed over time.

The multiple sensors also provide the ability to have more than one type of sensor, and up to six sensors in certain embodiments on the device of the same type, and between two and four sensors in alternative embodiments, which allows for summation of the signal or summation to the pressure points being derived. The summation allows averaging of the signal. The averaging of the signal allows for a more even distribution of the data set and increased confidence in the accuracy of the data.

While the invention has been described in terms of various specific embodiments, those skilled in the art will recognize that the invention can be practiced with modification.

What is claimed is:

1. An implantable medical device comprising:
a tubular housing defining a passage between a proximal end and a distal end of said housing, said passage providing fluid communication through said housing; and
a sensing unit positioned within said passage and coupled to said housing, said sensing unit configured to sense at least one of a physical, chemical, and physiological parameter within said passage.

2. An arterial pressure sensor device in accordance with claim 1 wherein said housing further comprises:
a distal retainer coupled to said housing at a midsection of said housing; and
a proximal retainer initially coupled to said housing at said proximal end, with said housing positioned within a tissue, said proximal end contacts a skin surface and said distal retainer contacts a vessel wall to retain said housing positioned within the tissue.

3. An implantable medical device in accordance with claim 1 wherein said housing is affixed within a tissue using a biocompatible adhesive including one of an acrylic-based adhesive, an epoxy-based adhesive, a polyurethane-based adhesive, and a silicon-based adhesive.

4. An implantable medical device in accordance with claim 1 wherein said sensing unit comprises at least one sensor coupled to said housing.

5. An implantable medical device in accordance with claim 4 wherein said at least one sensor comprises one of a pressure sensor, an optical sensor, a biochemical sensor, a protein sensor, a motion sensor, an accelerometer, a gyroscope, a temperature sensor, a chemical sensor, a pH sensor, and a genetic sensor.

6. An implantable medical device in accordance with claim 4 wherein said at least one sensor is fabricated using a microelectromechanical systems (MEMS) technology.

7. An implantable medical device in accordance with claim 1 further comprising a void formed in an inner surface of said housing, said void configured to receive a corresponding sensor of said at least one sensor.

8. An implantable medical device in accordance with claim 7 wherein said sensor is coupled within said corresponding void to said inner surface using one of an acrylic-based adhesive, an epoxy-based adhesive, a polyurethane-based adhesive, and a silicon-based adhesive.

9. An implantable medical device in accordance with claim 7 wherein said at least one sensor is coupled to said housing using one of a chemical bonding process, a heat bonding process, a soldering process, a suturing process, and an outer packaging material.

10. An implantable medical device in accordance with claim 1 wherein said sensing unit is configured to facilitate obtaining data for at least one of arterial pressure analysis, temperature analysis, blood chemical analysis, blood osmolar analysis, and cellular count analysis, said sensing unit further configured to generate and transmit measurement data wirelessly to an external receiver.

11. An implantable medical device in accordance with claim 1 wherein said sensing unit further comprises a substrate and at least one sensor coupled to said substrate.

12. An implantable medical device in accordance with claim 1 wherein said sensing unit is removably positioned within said housing.

13. An arterial pressure sensor device comprising:
a tubular housing defining a passage between a proximal end and a distal end of said housing, said passage providing fluid communication through said housing; and
a sensing unit positioned within said passage and coupled to said housing, said sensing unit configured to sense a pressure within said passage corresponding to an arterial pressure.

14. An arterial pressure sensor device in accordance with claim 13 wherein said housing further comprises:
a distal retainer coupled to said housing at a midsection of said housing; and
a proximal retainer initially coupled to said housing at said proximal end.

15. An arterial pressure sensor device in accordance with claim 14 wherein with said housing positioned within a tissue, said proximal end contacts a skin surface and said distal retainer contacts a vessel wall to retain said housing positioned within the tissue.

16. An arterial pressure sensor device in accordance with claim 14 wherein said distal retainer is positioned within a vessel and contacts an inner surface of a wall of the vessel, with said distal retainer positioned within the vessel, said housing provides fluid communication between the vessel and an external device.

17. An arterial pressure sensor device in accordance with claim 16 wherein said external device comprises one of a medical instrument positioned within said passage and a tubing operatively coupled to said proximal end of said housing.

18. An arterial pressure sensor device in accordance with claim 14 wherein said proximal retainer is movable with respect to said distal retainer to facilitate retaining said housing positioned within the tissue.

19. An arterial pressure sensor device in accordance with claim 18 wherein said proximal retainer is one of slidably movable and rotationally movable along a length of said housing with respect to said distal retainer.

20. An arterial pressure sensor device in accordance with claim 18 further comprising a first plurality of teeth formed on an outer surface of said housing and at least one second tooth formed on said proximal retainer cooperating with one tooth of said first plurality of teeth to adjustably position said proximal retainer with respect to said distal retainer.

21. An arterial pressure sensor device in accordance with claim 18 wherein said proximal retainer is adjustably positioned with respect to said distal retainer to apply a compressive force on the tissue to retain said housing positioned within the tissue.

22. An arterial pressure sensor device in accordance with claim 13 wherein said housing is affixed within a tissue using one of an acrylic-based adhesive, an epoxy-based adhesive, a polyurethane-based adhesive, and a silicon-based adhesive.

23. An arterial pressure sensor device in accordance with claim 13 wherein said sensing unit comprises at least one sensor coupled to said housing.

24. An arterial pressure sensor device in accordance with claim 23 wherein said at least one sensor comprises one of a pressure sensor, an optical sensor, a biochemical sensor, a protein sensor, a motion sensor, an accelerometer, a gyroscope, a temperature sensor, a chemical sensor, a pH sensor, and a genetic sensor.

25. An arterial pressure sensor device in accordance with claim 23 further comprising a void formed in an inner surface of said housing, said void configured to receive a corresponding sensor of said at least one sensor.

26. An arterial pressure sensor device in accordance with claim 25 wherein said at least one sensor is coupled within a corresponding void to said inner surface using one of an acrylic-based adhesive, an epoxy-based adhesive, a polyurethane-based adhesive, and a silicon-based adhesive.

27. An arterial pressure sensor device in accordance with claim 23 wherein said at least one sensor is coupled to said housing using one of a chemical bonding process, a heat bonding process, a soldering process, a suturing process, and an outer packaging material.

28. An arterial pressure sensor device in accordance with claim 23 wherein said at least one sensor is fabricated using a microelectromechanical systems (MEMS) technology.

29. An arterial pressure sensor device in accordance with claim 13 wherein said sensing unit is configured to facilitate obtaining data for at least one of arterial pressure analysis, temperature analysis, blood chemical analysis, blood osmolar analysis, and cellular count analysis, said sensing unit further configured to generate and transmit measurement data wirelessly to an external receiver.

30. An arterial pressure sensor device in accordance with claim 13 wherein said sensing unit further comprises a substrate and at least one sensor coupled to said substrate.

31. An arterial pressure sensor device in accordance with claim 13 wherein said sensing unit is removably positioned within said housing.

32. An implantable medical device comprising:
a tubular conduit defining a passage between a proximal end and a distal end of said conduit, said passage providing fluid communication through said conduit; and
a sensing unit positioned within said passage and coupled to said conduit, said sensing unit comprising at least one sensor configured to sense at least one of a physical, chemical, and physiological parameter to facilitate obtaining diagnostic data, said sensing unit configured to transmit the diagnostic data wirelessly to an external receiver.

33. An implantable medical device in accordance with claim 32 wherein said at least one sensor comprises at least one of a pressure sensor, an optical sensor, a biochemical sensor, a protein sensor, a motion sensor, an accelerometer, a gyroscope, a temperature sensor, a chemical sensor, a pH sensor, or a genetic sensor.

34. An implantable medical device in accordance with claim 32 wherein said sensing unit further comprises a substrate, each sensor of said at least one sensor coupled to said substrate.

35. An implantable medical device in accordance with claim 32 wherein said sensing unit is positioned within said conduit by an endovascular process.

36. A method for fabricating an implantable medical device, said method comprising:
fabricating a tubular housing defining a passage between a proximal end and a distal end of the housing;
positioning a sensing unit within the passage; and
coupling the sensing unit to the housing, the sensing unit configured to sense at least one of a physical, chemical, and physiological parameter within the passage.

37. A method in accordance with claim 36 wherein coupling the sensing unit to the housing comprises coupling at least one sensor of the sensing unit to the housing.

38. A method in accordance with claim 37 wherein the at least one sensor is positioned within a corresponding void formed in an inner surface of the housing.

39. A method in accordance with claim 38 wherein the sensor is coupled within the corresponding void to the inner surface using one of an acrylic-based adhesive, an epoxy-based adhesive, a polyurethane-based adhesive, and a silicon-based adhesive.

40. A method in accordance with claim 38 wherein the at least one sensor is coupled to the housing using one of a chemical bonding process, a heat bonding process, a soldering process, a suturing process, and an outer packaging material.

41. A method in accordance with claim 36 wherein the sensing unit is removably positioned within the housing.

42. An implantable device comprising a housing, said housing comprising:
a first sheet of material having a first surface defining a void therein, an electrically conducting surface patterned in said void formed in said first surface; and
a second sheet of material sealingly coupled to said first surface to enclose said void and form a hermetically sealed cavity, said second sheet of material patterned with electrically functional components, and said second sheet of material sealingly coupled to said first surface to form said housing defining a passage and a sensor configured to sense at least one of a physical, chemical, and physiological parameter within the passage.
